(19) United States
(12) Patent Application Publication
(10) Pub. No.: US 2007/0255595 A1
(43) Pub. Date: Nov. 1, 2007

(54) Pharmaceutical compound business methodology and software

(76) Inventor: Robert P. Nickell, Torrance, CA (US)

Correspondence Address: STETINA BRUNDA GARRED & BRUCKER, 75 ENTERPRISE, SUITE 250, ALISO VIEJO, CA, 92656, US

(21) Appl. No.: 11/820,960

(22) Filed: Jun. 21, 2007

Related U.S. Application Data
(60) Provisional application No. 60/894,164, filed on Mar. 9, 2007.

(51) Int. Cl. G06Q 10/00; A61B 5/00

(52) U.S. Cl. 705/2; 600/300

(57) ABSTRACT

A method for compounding pharmaceuticals, which includes placing an order for compounded pharmaceuticals, scheduling a date for the compounding, scheduling a location for the compounding, ordering the necessary chemicals required, providing the requested formulations, providing the actual compounding of the pharmaceuticals, and providing necessary equipment and staff for the compounding. A computer software program that facilitates the interaction between parties in a pharmaceutical compounding relationship. A portable pharmaceutical compounding lab that may be used in the method for compounding pharmaceuticals.

### CROSS-REFERENCE TO RELATED APPLICATIONS

This application claims the benefit of U.S. Provisional Application No. 60/894,164, filed on Mar. 9, 2007, the teachings of which are expressly incorporated by reference.

### STATEMENT RE: FEDERALLY SPONSORED RESEARCH/DEVELOPMENT

Not Applicable

### BACKGROUND

1. Field of the Invention

The present invention relates generally to pharmaceutical compounding, and more particularly to a business method for providing customized pharmaceutical compounding services to physicians and other licensed medical entities.

2. Background Art

Pharmaceutical compounding is the process of mixing drugs to fit the unique needs of a patient, usually performed by a pharmacist or a physician. One example where compounding can be extremely beneficial is when a patient is allergic to a certain dye and the mass produced version of a prescribed medicine contains the offending dye. In contrast to the manufactured medicine, a compounded version of the medicine can be prepared without use of the dye. Other examples where compounded medicines may be beneficial are patients requiring limited dosage strengths and smaller dosage forms (e.g., infants), patients with allergies to certain ingredients in mass produced drugs, veterinary medicine patients, and patients who need drugs that have been discontinued by pharmaceutical manufacturers. Pharmaceutical compounding may also be beneficial in allowing a physician to prescribe a medication containing a new compound that is currently being studied but is not yet used in commercially available medications.

There is an increasing demand for unique and customized compounded medications by physicians to allow for more personalized treatment of their patients as well as a demand for novel methods for enhancing and improving their revenue streams. It is within the standard of practice for physicians to employ, hire, or utilize any and all types of medical device aids, such as MRI units, X-ray units, casting, dispensing of repackaged medications, and compounding of pharmaceuticals. Although physicians in the United States may legally compound their own prescriptions, they typically do not have the skills or desire to do so. As such, there is a need for the providing of compounded medication services to physicians in small and large scale operations for onetime, semi-regular, and regular dispensing or in-office administration.

Physicians in the United States are legally authorized to compound pharmaceutical formulations themselves or to oversee and supervise the compounding of pharmaceutical formulations. Furthermore, physicians in the United States are legally authorized to contract with an outside service or agency to perform these compounding services performed under their supervision.

### BRIEF SUMMARY

One embodiment of the present invention is a portable compounding lab that can be delivered to a physician's office, set up, and taken down in a single day without disrupting the physician's practice. A team of compounding consultants arrives at the physician's office, assembles the portable lab, sets up the equipment, and compounds medications on behalf of the physician.

The compounding consultants, or service provider, will provide a staff of experts to conduct the compounding, including a consulting pharmacist, and multiple certified technicians all of which work under the supervision and licensure of the contracting physician. Although the amount of technicians present will depend on the size of the order, it is envisioned that there will normally be between two and six technicians on hand to compound the ordered medications. The service provider will also provide established formulations, assistance in creating new customized formulations based on experience and expertise, and the opportunity for the physician to create customized formulations of his or her choosing. The service provider will supply all necessary components on the day of compounding, including lab equipment, chemicals, an entire portable compounding lab, and all necessary staff.

The formulations may be compounded in any desired dosage and in a variety of forms, such as, but not limited to, ointments, creams, gels, transdermals, solutions, sprays, suspensions, tablets, caplets, pellets, patches, capsules, and any other sterile or non-sterile dosage delivery system either now known or created in the future. The service provider may also provide a computer software program that facilitates the interaction between the physician's office, the compounding consultants, the pharmacy chemical provider, and/or a medical management company. The software allows the physician to maintain inventory, place new orders, create custom formulations, track dispensing and administration, submit bills for collections, maintain transparency in accounting for claims submitted, as well as numerous other features.

One method envisioned by the present invention involves the use of a complete portable compounding lab (PCL) that can be set up and torn down in a single day. The PCL will be assembled in an empty area of a physician's office and includes, among other things, vinyl wipeable surface walls, air purifiers, powder hoods, small powder HEPA filters, large and small scale mixers, large and small electronic scales, at least one printer connected to the scales, glassware and stainless equipment necessary for pharmaceutical compounding, an ointment mill, a filling machine, heat unit, stir unit, pH modulator, modular cabinets, drawers, modular flooring, 220V electrical wiring if necessary, venting to the exterior if necessary, capsule machines, all chemicals including both active and inactive ingredients, bases and formulations. It is envisioned that the components present in the PCL may be added to or decreased depending on the particular order and needs of the physician.

Other embodiments may include a mobile compounding lab (MCL) wherein all of the necessary components are located within a motor vehicle, such as a bus, RV, truck, or trailer and the motor vehicle is transported to the physician's location. All compounding would then be conducted within the motor vehicle under the supervision and licensure of the physician but without requiring the set up and tear down of a PCL. This method may be of particular use if the physician does not have the available room in her office to set up a PCL. Another embodiment involves the designing, building, and staffing of a full-time compounding lab within the physician's office. This permanent lab would include all of the components of a PCL but would be a permanent fixture of the physician's office. All staffing and software support for the clinic would be provided by the compounding consultant. Compounding in this embodiment may be on a daily or semi-regular basis. Yet another embodiment would include a permanent compounding lab established in a location off-site from the physician. In this embodiment the physician would be transported to the off-site location and the compounding would occur under her supervision and licensure away from the physician's office.

The process begins with a physician ordering compounded formulations. The physician may order the compounds via a supplied computer software program. After the order has been submitted, labels are preprinted by the service provider and may include the name and address of the physician, the date the compound was prepared, the beyond use date, the lot number, the name of the compound, the strength, the directions, auxiliary labels, and a fill in the blank portion for the patient's name and date of service. The service provider may supply all of the necessary containers for the final products, a pharmacy cabinet to store and lock the medications prior to dispensing, all necessary compounding equipment, and the compounding staff. The physician is able to schedule the compounding via the computer software on a one-time or recurring basis, including weekly, bi-weekly, and monthly bases. Although the beyond use, or expiration date, of the compounds will typically be six months from the date of compounding, it is envisioned that no more than a thirty day supply will typically be compounded at a given time. As the physician dispenses the medications, the compound, patient's name, and demographic information may be entered into the computer software program and sent to the billing company for claim collection purposes. Both the physician and the compounding consultant may be remunerated upon the payment of the claim.

Four main components of the present invention include, but are not limited to, a computer software program, a compounding lab, compounding staff, and a quality assurance plan. The computer software program allows the physician to, among other things, place orders for upcoming compounding dates; schedule compounding dates by viewing a master calendar showing available dates; create new customized formulation orders; dispense and administer final compound medications to patients; maintain complete inventory records for dispensation, administration, returns, and re-orders; maintain a patient profile showing the date of service, specific patient information, name of compound, physician dispensing, quantity dispensed, and price charged; provide access to all claims accounting in process, including those paid, denied, or closed. The computer software program may optionally be combined with a barcode-scanner system to facilitate the dispensing and inventory control processes by scanning the barcode at dispensation rather than requiring manual entry. The computer software program may maintain patient demographic information that is obtained from the physician's database either by scanning, faxing, FTP; or direct manual entry of the information. The computer software may also interface with the claims and billing collection service and allows for the printing of bills on plain paper invoices and/or HCFA forms, including the list of chemicals and NDC numbers utilized, ICD-9 codes, and diagnosis. The software may also track reconciliation of claims and allow the physician to see an aging report for accounting. The same computer software may be utilized by the service provider to create labels for the final compounds, to maintain scheduling for compounding appointments, and to store and create customized compounded formulations.

In the portable variation of the compounding lab, a truck and team will typically be able to serve 8-10 physicians' offices on a regular basis. The trucks are designed to accept modular units, cabinets, and hydraulic carts, and to lock all of the equipment into place during transit. The truck may include a portable hot and cold water supply system for washing the compounding equipment and supplies. The truck may further include refrigerator and dishwasher units, a compressor for assisting with air circulation within the PCL, and/or a generator for providing 220V electricity to the PCL.

The compounding staff includes a lead technician and at least one assistant technician. A typical team will have three to six assistant technicians. The staff will further typically include a consulting pharmacist, a driver/handyman, and a support driver/dishwasher.

Quality assurance of the compounded products is met by multiple factors. In particular, the order may be placed online by the physician directly through the computer software interface while capturing an electronic signature. The order is then converted to labels and a list itemizing all compounds to be prepared is printed and emailed to the physician. The compound order is further reviewed by the consulting pharmacist against the formulations contained within the software database. Prior to beginning the compounding on the scheduled day, the physician will sign an acknowledgement form for the work to be performed as well a document indicating receipt of the necessary chemicals. During the compounding process, a log will be kept including the compound formulations, list of chemicals used, and the names of staff assigned to each compound. At the conclusion of the compounding, the physician will sign-off that the order is complete to her satisfaction and verify that all ordered compounds have been prepared. A random sample of each compound made will be taken and sent to an independent laboratory for stability and potency testing. When the testing data is available, the results will be scanned and made available through the computer software program. Optionally, the physician may be notified of the results via email.

The methods of this invention are designed to service physician offices, clinics, licensed pharmacies, hospitals, skilled nursing facilities, and other licensed and valid areas where compounding may be legally performed, and it is to be noted that all references herein to physician or physician's office is intended to include all of the previous categories. It is envisioned that this method may be utilized for practices located throughout the United States and in every type of specialty and practice for medicine currently practiced and contemplated in the future.

One market that would benefit greatly from pharmaceutical compounding as described in the present invention includes, but is not limited to, the workers' compensation market. This market covers physicians, physician practices, clinics, and medical groups that are caring for and accepting patients covered by workers' compensation insurance into their practice. Most of these physicians are looking for means to enhance their revenues that offer low risk. These clinics and physicians may benefit from having compounded medications prepared in their own facility, medical suite, clinic, or office that could then be dispensed directly to their patients. The physicians in this market may also benefit from having compounded medications prepared in an offsite location under their supervision. In either case, once the compounded medication is dispensed, the physician then has the right to submit a claim to the workers' compensation carrier for reimbursement.

Another market that would benefit from pharmaceutical compounding as described in the present invention includes, but is not limited to, the cash market for physicians and clinics. Physicians, who may range in specialty from OB/GYN, endocrinologists, internists, cosmetic surgeons, veterinarians, and others, could utilize pharmaceutical compounding to differentiate their practices. These physicians may desire to offer their own lines of compounded pharmaceuticals but do not have the expertise, know-how, or ability to get started. Specific compounds that may be of particular interest in this market may include bio-identical hormone replacement therapy, age management, cosmeceuticals, domesticated animal needs, podiatry, pain management, orthopedics, and neuropathics. In this market, the physicians would be reimbursed directly from their patients.

Yet another third market for pharmaceutical compounding as described in the present invention includes, but is not limited to, licensed retail or hospital pharmacies, other hospital practices, and larger multi-specialty clinics. In these situations, compounding would occur under proper licensure and authority of a physician and could occur on a regular basis, as often as daily.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a flowchart showing the general procedures of one embodiment of the present invention;

FIG. 2 is a flowchart showing more detailed procedures of FIG. 1;

FIG. 3 is a flowchart showing compounding processes of one embodiment of the present invention;

FIG. 4 is a flowchart showing dispensation of compounds in one embodiment of the present invention;

FIG. 5 is an external elevation view of a portable compounding lab being delivered to a medical office as envisioned in one embodiment of the present invention;

FIG. 6 is an elevation view of an empty space in a doctor's office prior to the assembly of the portable compounding lab of FIG. 5;

FIG. 7 is an elevation view of the installation of the portable compounding lab's temporary sidewalls in the empty space of FIG. 6;

FIG. 8 is an elevation view of the assembled portable compounding lab's sidewalls;

FIG. 9 is an elevation view of the completely assembled portable compounding lab including equipment; and

FIG. 10 is a cutaway view of FIG. 9 illustrating various equipment utilized in the compounding process.

### DETAILED DESCRIPTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

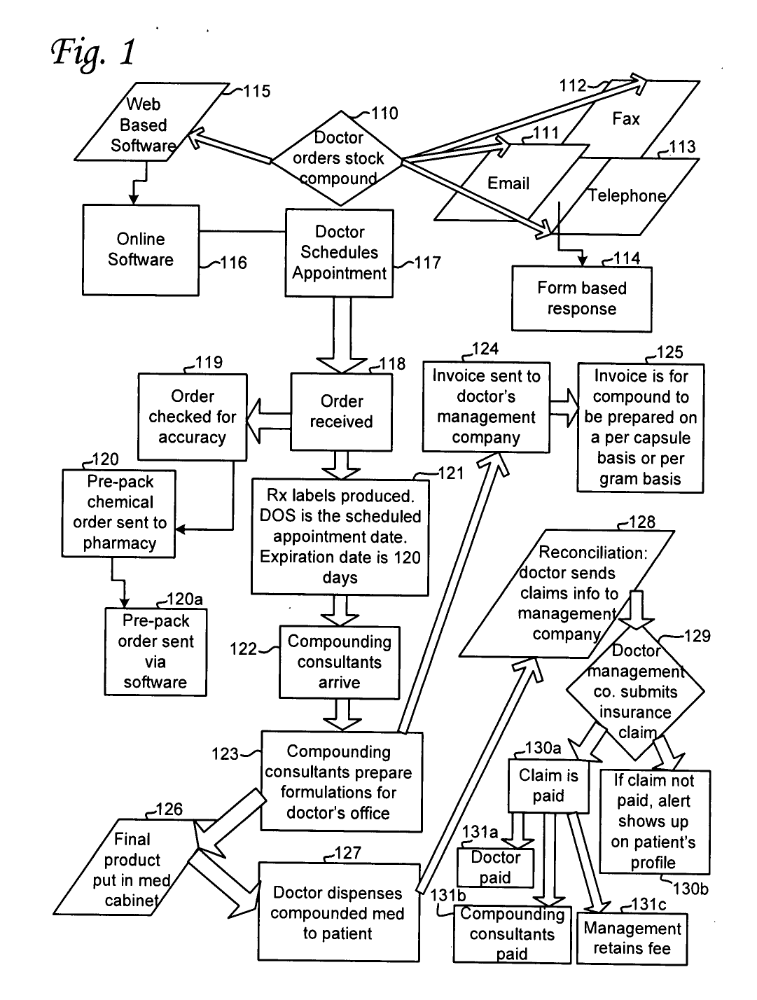

The present invention envisions novel methods for compounding pharmaceuticals under the supervision of a licensed physician as well as software for facilitating said methods. The methods utilize a service provider, or compounding consultant, who will do the actual compounding of pharmaceuticals for the contracting physician. The steps utilized in the general concept of the invention can best be seen in the flowchart of FIG. 1. The first step is for the doctor intending to use the invention to order the stock compounds in step 110. These compounds may be ordered by a variety of methods including, but not limited to, via email 111, facsimile machine 112, or telephone order 113. All of these methods may include a form-based response 114 to place the orders, wherein the form may include a checklist of available compounds to order. The form may also allow for the creation of a new compound whereby the ordering doctor indicates the desired components and amounts of each component within the compound. The form may further include an option to repeat the previous order if the doctor has previously used the service envisioned by the present invention. The details of the order form will then be inputted into a computer software program, a description of which will be provided below, by an employee of the provider.

Alternatively, the doctor may place the order by instead logging directly onto the online computer software program via a global network system as shown in step 115. The online software 116 features a plurality of benefits in placing the orders. For example, the online software 116 may present a listing of all compounds currently available, an interactive tool for creating a new compound based on active ingredients and proportions of each, the ability to repeat a previously stored order with or without the addition and/or deletion of specific compounds, the ability to repeat a previously stored order with changes in quantity of specific compounds, the ability to choose a specific desired inventory balance of certain compounds with an auto-refill option wherein once a bottom level of inventory of the compound is reached in the doctor's possession an automatic refill order will be placed. In this step, once the order is placed via the online software 116, the order is verified by the software program and asks the doctor to agree to certain required terms, upon the doctor's agreement, the order is automatically forwarded to the provider to fill.

In step 117, the doctor schedules an appointment for the compounding to occur on the provider's master calendar. This may be achieved directly online wherein the doctor can immediately view the available days for compounding and select the optimal day, or the scheduling may occur via a telephone call or email wherein a representative of the provider lists the available upcoming dates and the doctor chooses from one of the presented dates.

The order is received by the provider in step 118 and checked for accuracy in step 119. After the accuracy has been verified, a pre-pack chemical order is sent to a pharmacy in step 120 to obtain the necessary active ingredients for the requested compounds. The necessary active ingredients may be determined by a listing of ingredients contained within the formulary software. The pre-pack order may be sent to the pharmacy directly via software in step 120a. This allows the pharmacy to print the order at their own location, to print the necessary labels for the pre-pack order, and to print the transfer form authorizing transfer of the medical chemicals directly to the doctor's office. Further, the pharmacy may optionally send the invoice for the chemicals to the provider for collections on behalf of the doctor, or may instead directly send the invoice to the doctor's office. Finally, the pharmacy will pre-pack, box, and seal the order and deliver the medical chemicals directly to the doctor's office on the scheduled compounding date. By directly delivering the restricted medical compounds to the doctor's office, the method prevents the provider from needing a licensed physician to accept delivery of restricted compounds.

After the order has been received in step 118, the provider will print labels for the final compounds to be produced in step 121. The date of service (DOS) printed on the label is the appointment date scheduled in step 117. Although the longevity of the compounds can vary and are typically long-lasting, it is envisioned that the labels will carry an expiration date of 120 days from the DOS date.

On the date scheduled in step 117, the compounding consultants will arrive at a pre-arranged location in step 122. Although this location will typically be at the physician's office, there are numerous locations that this method may employ which will be discussed in greater detail below. In step 123, the compounding consultants prepare the ordered formulations for the doctor's office. The final products are then placed in a locking medicine cabinet for delivery to the physician in step 126. After having received the formulations, the doctor may then dispense the compounded medications to his or her patients as necessary in step 127. It is to be noted that at all times the compounded formulations are under the control of the physician and all compounding is conducted under the supervision and licensure of the physician.

After having filled the order for the doctor, the compounding consultant will send an invoice to the doctor's management company in step 124. The invoice in step 125 includes a detailed description of the products delivered to the doctor and the price, which may be prepared on a per capsule or per gram basis, includes many factors including the cost of the raw ingredients, the time necessary to compound the formulas, the skill required to prepare the formulations, and other relevant factors.

After dispensing a formulation in step 127, the doctor may reconcile the transaction by sending claims information to the management company in step 128. The management company then submits a claim to the patient's insurance carrier in step 129. This step is not necessary, however, as the patient may choose to pay cash rather than have his or her insurance billed for the medication. If an insurance company is billed, the may choose to either pay the claim 130a or to deny the claim 130b. If the claim is denied, the management company may alert the doctor on the status of the patient. This status may then show up as an alert on the patient's profile within the computer software. If the claim is paid, however, then the doctor will be reimbursed 131a, the compounding consultant service provider will be paid 131b, and the management company will typically retain a fee for offering its service 131c.

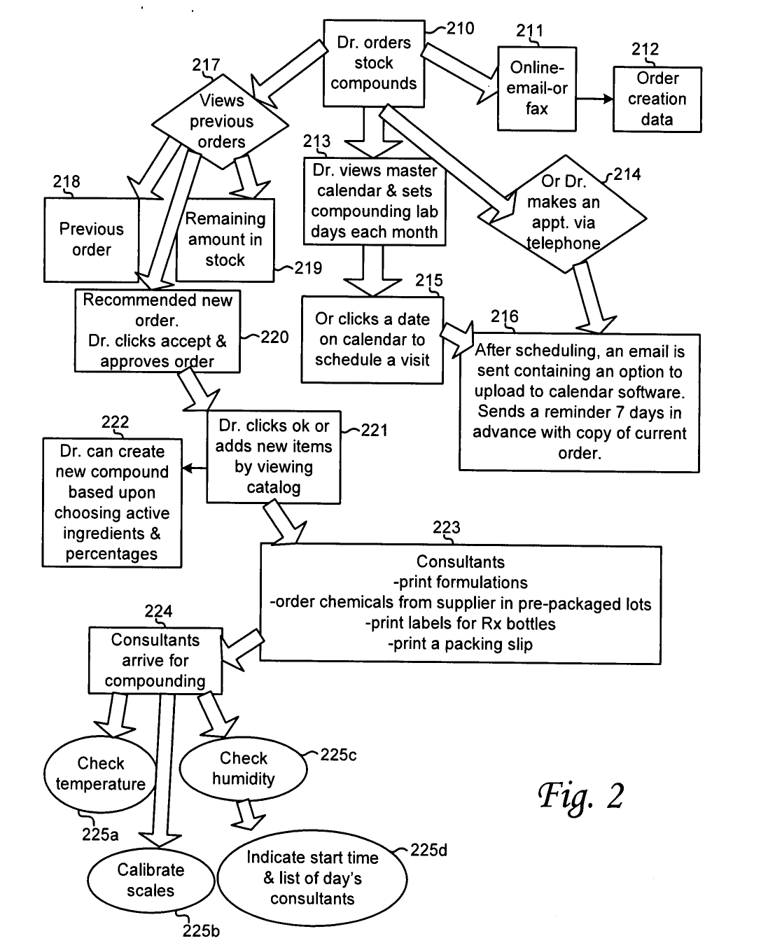

FIG. 2 is a flowchart which illustrates the ordering and compounding processes in greater detail than in FIG. 1. The process begins in step 210 when the doctor requesting the service orders stock compounds. Step 211 envisions that the ordering may occur via different mediums, including placing the order online via the computer software program, via an email message, or via the facsimile submission of an order form. The order data may be created in step 212 depending on the submission format. For example, the order may be created by utilizing a check box system in the computer software program based on previous order information, or a form may be downloaded and the desired order information may be written on the form and faxed back to the compounding consultants, or the doctor may submit an email message to the compounding consultants with the order information contained therein.

The doctor also may view a master calendar via the computer software program showing the available dates for compounding in step 213 and establish set compounding lab days each month on a recurring schedule. Alternatively, the doctor may make a phone call to the compounding consultants and set up a compounding date in step 214. The doctor may alternatively select a single date for compounding in step 215 by clicking on an open date in the computer software program's master calendar. After the compounding date is established by any of these methods, the compounding consultants may send a confirmation email to the doctor in step 216. The confirmation email may optionally include an option to upload the scheduled date to a computer calendar software program used on the doctor's computer. The compounding consultants may send a reminder email to the doctor containing a copy of the current order. The reminder email will be sent to the doctor on a predetermined date prior to the compounding, preferably seven days before the scheduled compounding date.

The doctor may also review previous orders via the online computer software program in step 217. While doing so, the doctor may view what compounds were ordered previously in step 218, may view the amount remaining of each compound in step 219, and may accept and approve a new order based on this information in step 220. The new order information presented in step 220 attempts to complete the previous order, that is, the amounts previously ordered for each compound less the amounts of each remaining in stock will be the pre-filled amounts for the new order. In step 221, the doctor may accept these pre-filled amounts by clicking “ok” in the computer software program or may add new items by viewing an online catalog, delete items, or change the amounts of items. In addition, in step 222 the doctor may choose to create new compounds based upon choosing active ingredients and percentages of each.

Once all of the order information has been submitted by the doctor, the compounding consultants will print the formulation orders in step 223, as well as order the chemicals from the supplier in pre-packaged lots under the agency of the doctor, print labels for the prescription bottles, and print a packing slip. The consultants then arrive on the scheduled day to compound the desired formulations in step 224. Prior to beginning the compounding, the consultants will perform various verifications to ensure the accuracy of the compounds such as, checking the temperature in step 225a, calibrating the scales in step 225b, checking the humidity of the lab in step 225c, and indicating the start time of the compounding and a list of consultants working that day in step 225d.

It is envisioned that various forms will be utilized during all stages of the process in order to facilitate the efficiency and accuracy of the process. These forms may include, but are not limited to, the following: formulations sheets, lists or order forms for the doctor to sign, pharmacist protocol forms, packing slips, lists of chemicals to pre-pack and ship, copies of the orders, temperature log sheets, humidity log sheets, scale calibration log sheets, staff attendance log sheets, manual order sheets, HIPPA agreements, new account setup sheets, recall notice forms, pharmacy to doctor transfer authorization forms, employment service contracts for the compounding consultants, and agency authorization forms for pre-pack order requests to pharmacy.

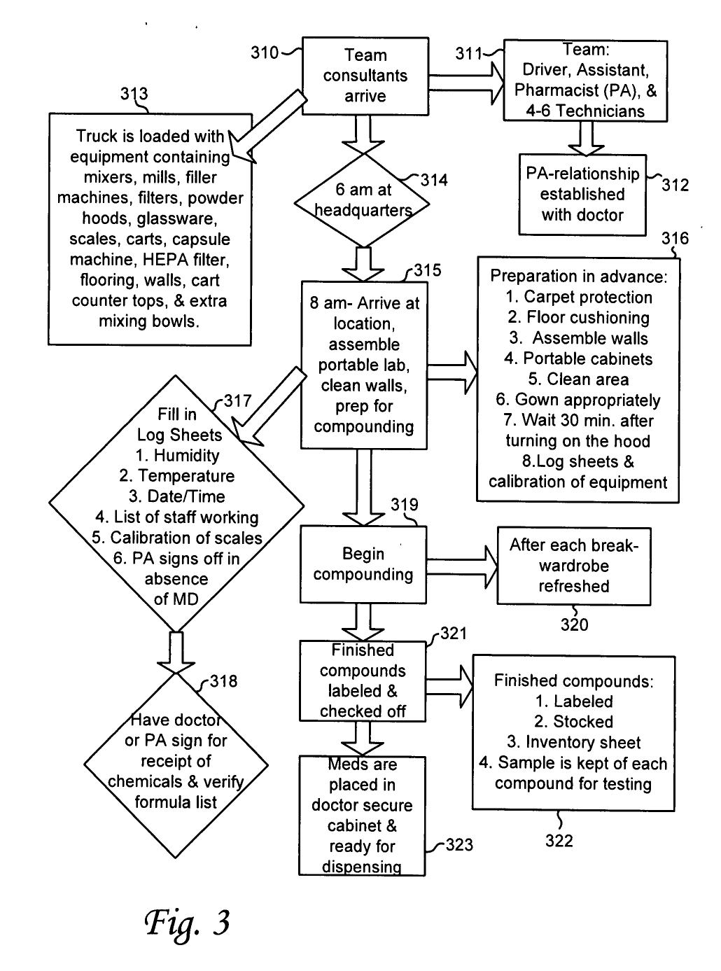

The flowchart of FIG. 3 illustrates certain variations of compounding once the consultants have arrived at the compounding location. In step 310, the compounding team arrives at the compounding location on the predetermined date. One embodiment of a compounding team is illustrated in step 311 consisting of a driver, an assistant, a pharmacist (PA), and four to six pharmacy technicians. The pharmacist establishes a relationship with the doctor in step 312 so that the pharmacist acts under the direction and supervision of the doctor.

The team typically arrives in a truck loaded with all of the necessary equipment to complete the day's compounding. Step 313 illustrates some of the equipment that may be loaded on the truck, which may include, but is not limited to, the following: mixers, mills, filler machines, filters, powder hoods, glassware, scales, carts, capsule machines, HEPA filters, flooring, walls, cart counter tops, and extra mixing bowls. Although the arrival time will depend on the amount of formulas to be compounded and the needs of the contracting doctor, the typical start time for the compounding team is illustrated in step 314 as being 6:00 am. The team will then typically arrive at the predetermined compounding location by 8:00 am and begin to assemble the portable lab within the compounding location in step 315. The team also cleans the walls and takes all necessary steps to prepare for the day's compounding.

Some of this preparation is shown in step 316 and includes protecting the building's carpet with temporary plastic, followed by laying down a temporary smooth cushioned flooring, assembling the temporary compounding lab walls, bringing portable cabinets into the assembled temporary lab, cleaning all counters and walls, and mopping the floor. Prior to beginning any compounding the consulting team will dress appropriately in surgical gowns to prevent any contamination. Further, the powder hoods, or air units, are turned on at least 30 minutes prior to the commencement of any compounding to ensure a clean-room status and adequate ventilation in the temporary lab. Finally, all equipment is properly calibrated and log sheets filled out.

The log sheets are filled out in step 317 and include logging the humidity, temperature, the date and start time, a listing of the staff present, the calibration of the scales and other equipment, and a final approval prior to the commencement of compounding that is signed by the contracting doctor or by the pharmacist in the absence of the doctor. In step 318, either the contracting doctor or the pharmacist authorizes the receipt of restricted chemicals and verifies the formula list.

At this point, the team is ready to commence compounding in step 319. During the day as breaks are taken, the team must re-gown prior to compounding again in step 320. The re-gowning includes the replacement of the gowns, gloves, hats, and masks. As the day progresses and compounds are finished, they are labeled and checked off on the compounds list in step 321. The finished compounds are labeled and stocked in a medicine cabinet, an inventory sheet containing all of the prescription numbers and names of the medications compounded is left with the doctor, and a sample of each compound is retained by the consultants for further potency testing in step 322. All of the compounded medications are placed in a secure cabinet accessible only by the doctor in step 323 and are now ready for dispensing.

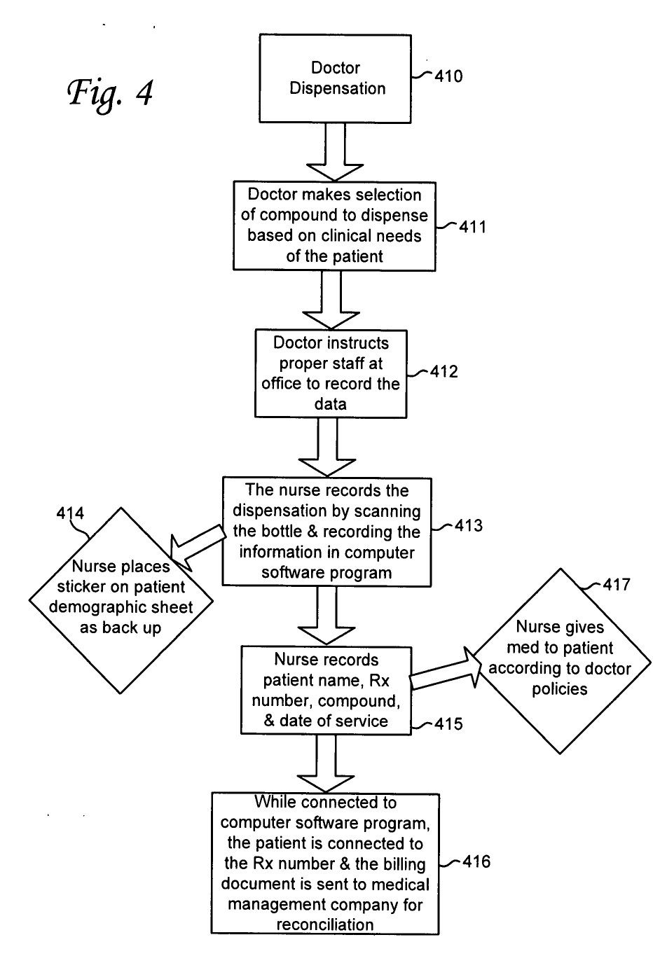

FIG. 4 illustrates a flowchart of the dispensation process after the formulations have been compounded. The process begins with the doctor dispensation in step 410. The doctor selects the appropriate compound to dispense based on the clinical needs of the patient in step 411. Next, the doctor instructs the proper staff in step 412 to record the dispensation data. The nurse then records the dispensation in step 413 and records the information in the computer software program. The dispensation may be recorded by scanning the code on the label or by inputting the prescription number manually. The nurse may then optionally place a prescription sticker on the patient demographic sheet as a back up recordation of the dispensation. The nurse then records the patient's name, prescription number, the name of the compound, and the date of service in step 415. In step 416, the computer software program automatically connects the prescription number to the patient and the proper billing statement is sent to the medical management company for later reconciliation. The nurse may then physically transfer the medication to the patient pursuant to the doctor's policies in step 417.

FIG. 5 illustrates the initial delivery of the portable compounding lab at the predetermined compounding location 10 via an equipment truck 12. The truck transports all the necessary equipment and is specialized for the transportation and further compounding requirements. The truck may include built in cabinets for storing compounding equipment and reagents. One embodiment of the truck includes a 24 foot box for transporting the equipment. A non-tilt lift gate is used in order to load and unload the compounding equipment. The truck may further include plumbing for running hot and cold water, if necessary, and may include at least one deep sink for washing the labware and cleaning up equipment. The truck may also include a built-in refrigerator and/or dishwasher and a generator for backup power. The truck may be 220V capable for running heavy duty equipment, if necessary. Another option for the truck is satellite radio with speakers throughout the truck for the worker's entertainment during the day and while on break. The bed of the truck may be made of a vinyl flooring that is easily cleanable. The truck may also include lock-in portions that are custom designed to retain the portable counter carts and hydraulic devices during transport. For the comfort of the team, the truck may also include folding chairs for break periods and full air conditioning and heating.

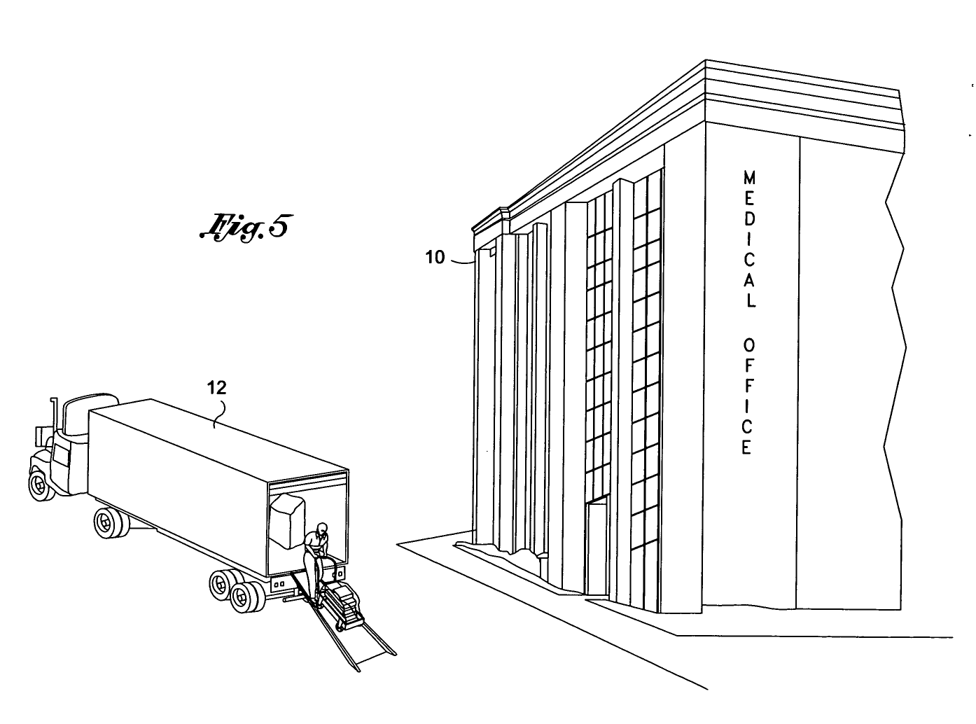

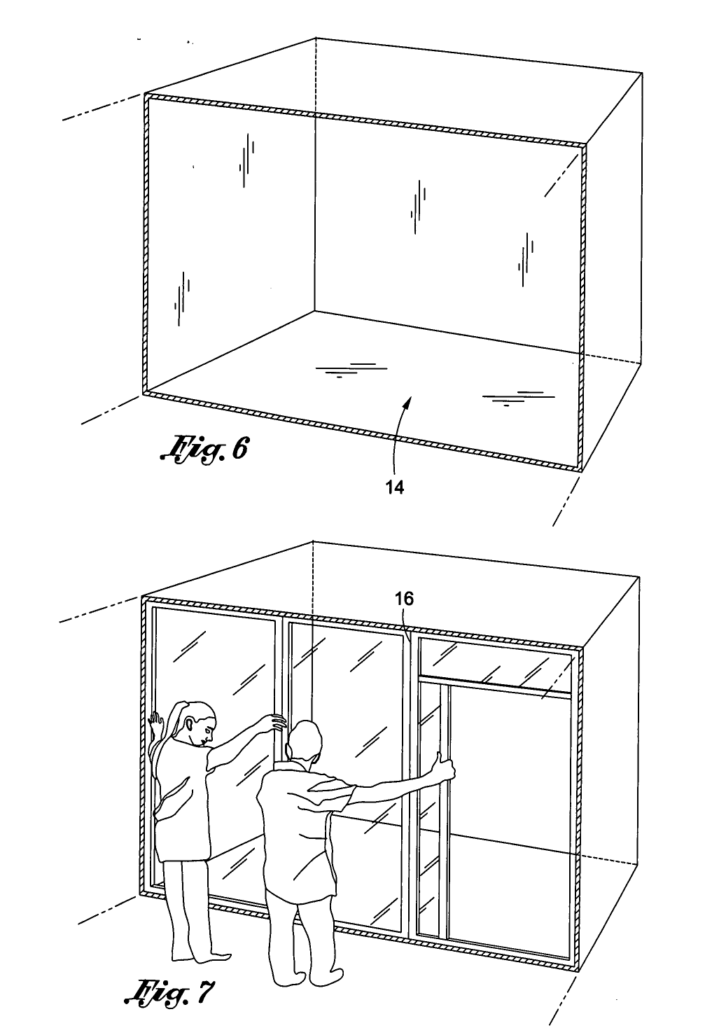

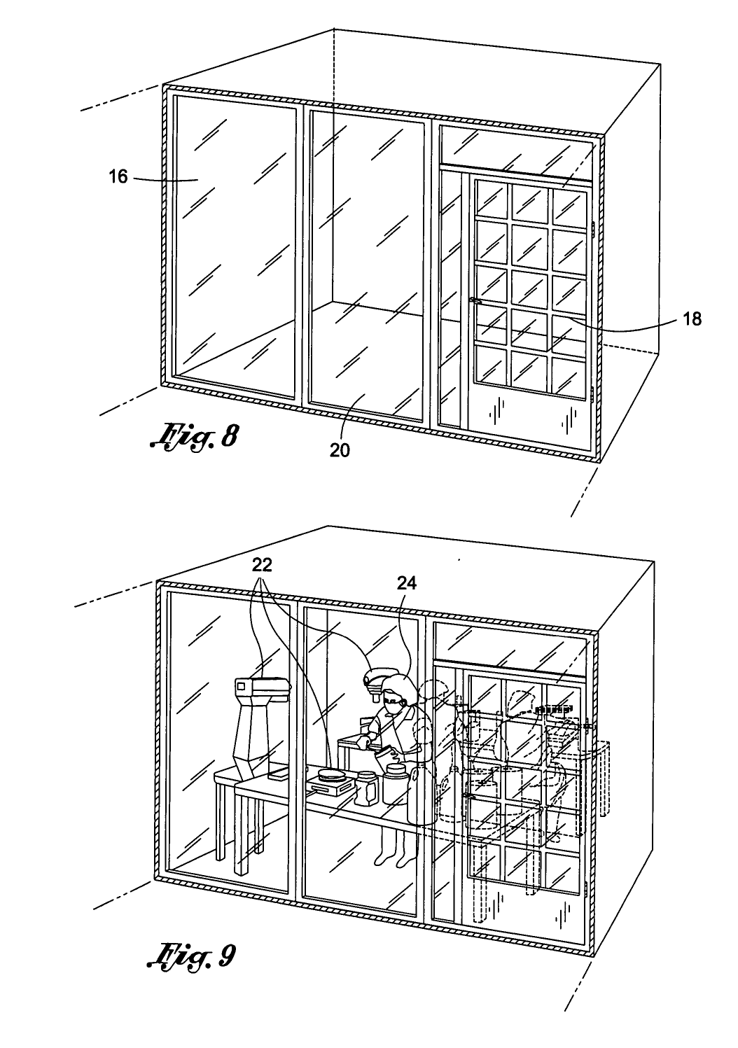

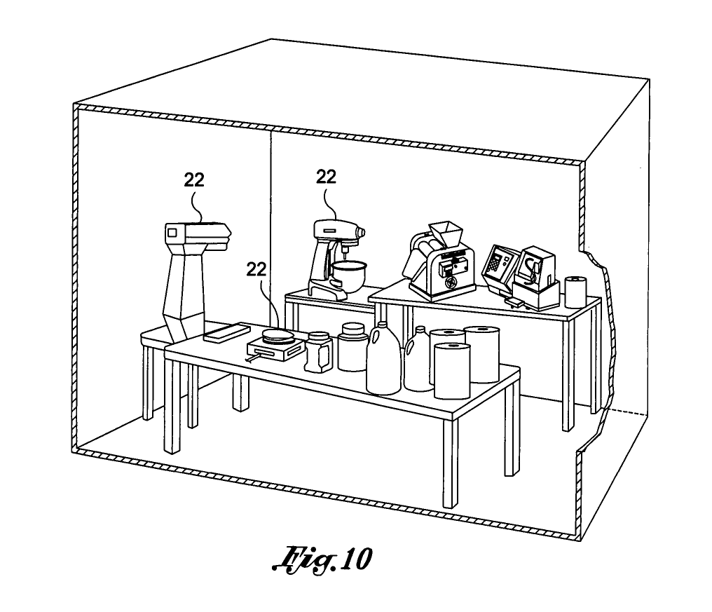

FIG. 6 illustrates an empty space 14 within the compounding location 10 where the temporary compounding lab will be assembled. FIG. 7 illustrates the assembly of the outer walls 16 of the portable compounding lab. FIG. 8 is an illustration of the fully assembled portable compounding lab skeleton, including the temporary walls 16, an entry door 18, and temporary flooring 20. FIG. 9 illustrates a fully assembled temporary compounding lab in full operation with all necessary compounding equipment 22 and compounding specialists 24. The assembled compounding lab is fully FDA compliant and the staff is fully qualified to perform their specific compounding duties. FIG. 10 shows the equipment utilized in the compounding process in greater detail.

Although the embodiment illustrated in the figures are of an in-office compounding lab, it is envisioned that the temporary compounding lab may be assembled in various locations, for example, in a mobile compounding lab that is brought to the doctor's office and wherein all of the compounding is performed within the mobile lab outside of the doctor's office. This mobile compounding lab may be a converted motor home, bus, or truck. The vehicle is FDA compliant and includes wipeable walls and floors, HEPA filters, full air conditioning and heating, built-in counters with bacteriostatic tops, sterile hoods in which the actual compounding is performed, and a full set of supplies and compounding equipment as in the in-office embodiment described above. The compounding lab may also be in a centrally located permanent location where a doctor, or his agent, may come to authorize the required compounding.

Another aspect of the present invention is the computer software program that facilitates the interaction between the doctor's office, the compounding consultant service provider, the chemical house of the pharmacy, and the medical management company. The computer software of the present invention allows the doctor's office to order necessary compounds via a global network. The ordering may be achieved by repeating a previous order. Additionally, there may be an option to auto-order refills based on a preset inventory minimum. Another option allows the doctor to custom order compounds based on active ingredients and the proportions of each. The computer software program also allows the doctor's office to view and/or print expired drug reports and/or current inventory reports. The doctor may also choose to submit weekly “cures” data and to order compounds by name, strength, quantity, number of pills, and number of bottles. The ordering may be completed by choosing these options via onscreen checkboxes and may also submit additional instructions. Once the doctor submits the order, a copy is emailed to the doctor's email address of record and to the compounding consultant's representative for this particular doctor. The doctor may then schedule the appointment for compounding by viewing the consultant's master calendar online and choosing an available date. The online program also allows the doctor to make referrals of the program to new doctors, to view reconciliation reports, and to upload the scheduled date to a calendar software program on the doctor's computer.

The scheduling may be achieved by viewing a master calendar through the computer software program via a global network and to select an available day. The unavailable days may be differentiated from available days by darkening the unavailable days or indicating the status by some other manner. It is envisioned that the doctor may schedule compounding appointments up to six months in advance, including recurring appointments. If the doctor has scheduled recurring appointments, a notice may be sent to the doctor 5 months in advance of the last appointment to allow the doctor to schedule new appointments. The compounding consultants may institute a cancellation policy, such as where a $500.00 cancellation fee will be charged to the doctor unless she cancels at least a week in advance of the scheduled date. Any changes made to the order or the date of the compounding appointment will cause an update email to be sent to the doctor. Upon scheduling an appointment, the doctor must agree to all terms and conditions established by the service provider, and she has the ability to have the scheduled appointments uploaded to a calendar software program. The doctor's phone number and email address may automatically be uploaded to the schedule and if there is a controlled substance as an active ingredient in any of the ordered compounds, the compound may be highlighted and the DEA number of the doctor will be printed on all forms next to the signature block of the physician.

On the service provider's end, after the doctor has placed an order it is received by the service provider. Based on the order, the computer software program creates a pre-pack chemical list based on formulations within the system. The pre-pack chemical list includes the appointment date, the name of the doctor, address of the doctor, and the doctor's DEA license number. A preview screen of the order is viewed by a service provider technician, if the order is acceptable labels are printed and the pre-pack chemical list is forwarded to a pharmacy. If the order is not acceptable, the technician makes necessary changes, prints the labels, and forwards the order to the pharmacy. The date of service (DOS) on the labels is the scheduled compounding date. Once the labels are printed, a copy of the final order sheet is printed and emailed to the doctor and contains the scheduled compounding date as a reminder.

At the same time, the pharmacy pre-packs all of the ordered chemicals and delivers them to the doctor's office, or other predetermined compounding location, on the scheduled compounding date. The computer software program also allows the doctor to notify the compounding consultant with problems on the compounds, for the consultants to notify the doctor of any recalls, and for the consultants to post potency studies from the test samples retained during previous compounding appointments.

When the medications are dispensed, the doctor may upload patient demographic information through the computer software program to the compounding consultants system. The patient name is then linked to the prescription number, either by scanning the label code or by manual entry. The patient date entered and transmitted may include the patient's first name, last name, date of birth, social security number, date of service, patient demographic information, and prescription number. Once the information is uploaded, the data is sent directly to the billing management company. Once all of this has been done, the doctor is able to generate numerous reports, including cures reports, patient reports, daily reports, amount billed reports, and transmission reports.

The computer software program also allows for reconciliation of the billing. Access to at least this portion of the computer software may be password protected. This portion of the program allows the viewing of the aging of the accounts receivable and shows the patient name, prescription number, date of birth, amount billed, date paid, amount paid, and any variance. The management company may also print invoices which include the ingredient list from compounds by linking to the prescription number in the computer software program and send them to insurance carriers. The management company may also reconcile payments online and can send notes back to the doctor with regard to certain patient pay history that shows on the doctor's screen at the time of dispensation.

The methods of the present invention are designed to meet all government regulations in place in the United States. For example, the pharmacy chemical company will request a copy of the doctor's licensure and her DEA registration. The pharmacy will also require a copy of the agency agreement between the compounding consultants and the doctor prior to transferring pre-pack chemical orders. The pharmacy will also provide pre-pack labels that include the lot number, date of fill, and the quantity and will provide a packing slip and inventory sheet for all delivered compounds. The pharmacy will also require a copy of the actual pre-pack order, will require an inventory return sheet for chemicals sent to the doctor but not used during the compounding, and will present an invoice to the compounding consultants as agents paying on behalf of the doctor.

In the doctor's office, the doctor and the service provider pharmacist will sign a compounding protocol wherein the pharmacist acts under the agency of the doctor. The doctor will also sign an employment contract with the compounding consultants. The contract will discuss services to be rendered and also provides for an agency agreement to forward orders on behalf of the doctor for pre-pack chemicals. If the compounding is done within the doctor's office, the doctor agrees that a properly ventilated location will be provided for the temporary compounding lab. HIPPA agreements to protect the patients' privacy will also be executed. The compounding consultants will then have agency authorization to transmit orders to the pharmacy chemical house for processing and to act as supervised compounders to perform services.

The medical management company will also be a party to certain contracts, namely with the doctor to pay invoices on behalf of the doctor, with the doctor to bill and collect claims for compounded prescriptions dispensed in the doctor's office, and a HIPPA agreement for protection of patients' privacy.

The above description is given by way of example, and not limitation. Given the above disclosure, one skilled in the art could devise variations that are within the scope and spirit of the invention disclosed herein, including various locations for performing the compounding. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A method for compounding pharmaceuticals, the method comprising the following steps:
a) receiving an order from a licensed physician for at least one compounded pharmaceutical;
b) scheduling a date for the compounding to occur;
c) scheduling a location for the compounding to occur;
d) arranging for chemicals required for the compounding of the pharmaceuticals to be provided on the scheduled date and at the scheduled location;
e) providing necessary equipment and staff on the scheduled date at the scheduled location for the compounding;
f) compounding the pharmaceuticals under the licensure of the physician

2. The method of claim 1, wherein the order in step (a) is placed via the submission of an email to a service provider.

3. The method of claim 1, wherein the order in step (a) is placed via the submission of a form to a service provider by facsimile machine.

4. The method of claim 1, wherein the order in step (a) is placed via a telephone call to a service provider.

5. The method of claim 1, wherein the order in step (a) is placed via data entry within a computer software program over a global network.

6. The method of claim 5, wherein the computer software program provides a listing of all currently available compounds.

7. The method of claim 5, wherein the computer software program provides a pre-populated form of previously ordered compounds.

8. The method of claim 5, wherein the computer software program allows for the creation of new compounds based on active ingredient proportions.

9. The method of claim 5, wherein the computer software program allows for the maintenance of a pre-set minimum inventory level.

10. The method of claim 1, wherein the compounding date is scheduled in step (b) via a telephone call to a service provider.

11. The method of claim 1, wherein the compounding date is scheduled in step (b) via a computer software program over a global network.

12. The method of claim 1, wherein the compounding date scheduled in step (b) is a recurring appointment.

13. The method of claim 1, wherein the compounding location scheduled in step (c) is in a portable compounding lab set up within the physician's office.

14. The method of claim 1, wherein the compounding location scheduled in step (c) is in a permanent compounding lab set up within the physician's office.

15. The method of claim 1, wherein the compounding location scheduled in step (c) is in a mobile compounding lab set up near the physician's office.

16. The method of claim 1, wherein the compounding location scheduled in step (c) is in a permanent compounding lab away from the physician's office and the physician is transported to the permanent compounding lab for the duration of the compounding in step (f).

17. The method of claim 1, wherein the chemicals are ordered in step (d) by a service provider based upon the active ingredients in the compounds ordered in step (a).

18. The method of claim 1, wherein the chemicals in step (d) are ordered and delivered to the scheduled compounding location of the scheduled compounding date.

19. The method of claim 1, wherein the staff in step (e) includes at least one compounding technician and at least one pharmacist.

20. The method of claim 1 further comprising the following steps:
g) dispensing the compounded pharmaceuticals to a patient requiring the same;
h) sending claim information to a medical management company.

21. A computer software program that facilitates the interaction between contracting parties in a pharmaceutical compounding relationship when compounding is provided on site comprising the functionality of;
a) receiving an order from a licensed physician for at least one compounded pharmaceutical;
b) scheduling a date for the compounding to occur;
c) scheduling a location for the compounding to occur;
d) arranging for chemicals required for the compounding of the pharmaceuticals to be provided on the scheduled date and at the scheduled location;
e) providing necessary equipment and staff on the scheduled date at the scheduled location for the compounding;

22. The computer software program of claim 21, wherein the parties include at least two parties selected from the group consisting of a physician's office, a compounding consultant service provider, a pharmacy chemical house, and a medical management company.

23. The computer software program of claim 22, wherein the parties interact through the computer software program over a global network.

24. The computer software program of claim 23, wherein the computer software program enables a physician's office to place an order for compounded pharmaceuticals from a service provider.

25. The computer software program of claim 23, wherein the computer software program enables a physician's office to schedule a date for compounding pharmaceuticals with a service provider.

26. The computer software program of claim 23, wherein the computer software program enables a service provider to order controlled chemicals from a pharmacy.

27. The computer software program of claim 23, wherein the computer software program enables a physician's office to submit billing information to a medical management company.

28. The computer software program of claim 23, wherein the computer software program enables a physician's office to receive billing information from a medical management company.

29. The computer software program of claim 21, wherein the computer software program enables a physician's office to maintain inventory of previously compounded pharmaceuticals.

30. The computer software program of claim 21, wherein the computer software program enables a physician's office to view reports related to compounded pharmaceuticals.

31. The computer software program of claim 21, wherein the computer software program enables a physician's office to print reports related to compounded pharmaceuticals.

32. A portable pharmaceutical compounding lab comprising:
temporary walls;
modular flooring;
at least one air purifier;
at least one powder hood;
at least one small powder HEPA filter;
at least one mixer;
at least one scale;
an ointment mill;
a filling machine;
a heat unit;
a stir unit;
and a pH modulator.

33. The compounding lab of claim 32, wherein the temporary walls are constructed from a vinyl.

34. The compounding lab of claim 32, wherein the mixers comprise at least one large scale mixer and at least one small scale mixer.

35. The compounding lab of claim 32, wherein the scales comprise at least one large scale and at least one small electronic scale.

36. The compounding lab of claim 32 further comprising at least one printer connected to the scales.

37. The compounding lab of claim 32 further comprising glassware and stainless equipment.

38. The compounding lab of claim 32 further comprising modular cabinets.

39. The compounding lab of claim 32 further comprising 220V electrical wiring.

40. The compounding lab of claim 32 further comprising a ventilation system.

41. The compounding lab of claim 32 further comprising capsule machines.

42. The compounding lab of claim 32 further comprising inactive ingredients for pharmaceutical compounds.
